(19) United States
(12) Patent Application Publication
(10) Pub. No.: US 2014/0261435 A1
(43) Pub. Date: Sep. 18, 2014

(54) CUSHION FOR PATIENT INTERFACE WITH LOCALIZED REGION OF REDUCED STIFFNESS

(71) Applicant: KONINKLIJKE PHILIPS N.V., EINDHOVEN (NL)

(72) Inventor: Justin Edward Rothermel, Monroeville, PA (US)

(21) Appl. No.: 14/351,359

(22) PCT Filed: Oct. 24, 2012
(86) PCT No.: PCT/IB2012/055848

§ 371 Date: Apr. 11, 2014

Related U.S. Application Data
(60) Provisional application No. 61/551,637, filed on Oct. 26, 2011.

(51) Int. Cl. A61M 16/06; A61M 16/00

(52) U.S. Cl. CPC A61M 16/0622; A61M 16/0683; A61M 16/0057
USPC 128/205.25

(57) ABSTRACT

An improved resilient cushion of a patient interface is structured to engage the face of a patient and includes a deformable bellows portion having a pair of deformable elements. One of the deformable elements has an indentation formed therein that provides a localized region of reduced stiffness and which results in an improved fit on the face of the patient.

### CROSS-REFERENCE TO RELATED APPLICATIONS

This patent application claims the priority benefit under 35 U.S.C. §119(e) of U.S. Provisional Application No. 61/551,637 filed on Oct. 26, 2011, the contents of which are herein incorporated by reference.

### BACKGROUND OF THE INVENTION

1. Field of the Invention

The present invention pertains to a patient interface for delivering a flow of breathing gas to a patient and, in particular, to an improved deformable cushion for a patient interface having formed therein an indentation that provides a localized region of reduced stiffness.

2. Description of the Related Art

There are numerous situations where it is necessary or desirable to deliver a flow of breathing gas non-invasively to the airway of a patient, i.e., without intubating the patient or surgically inserting a tracheal tube in their esophagus. For example, it is known to ventilate a patient using a technique known as non-invasive ventilation. It is also known to deliver continuous positive airway pressure (CPAP) or variable airway pressure, which varies with the patient's respiratory cycle, to treat a medical disorder, such as sleep apnea syndrome, in particular, obstructive sleep apnea (OSA), or congestive heart failure.

Non-invasive ventilation and pressure support therapies involve the placement of a respiratory patient interface device including a patient interface that is typically secured on the face of a patient by a headgear assembly. The patient interface may be, without limitation, a nasal mask that covers the patient's nose, a nasal cushion having nasal prongs that are received within the patient's nares, a nasal/oral mask that covers the nose and mouth, or full face mask that covers the patient's face. It is known to maintain such devices on the face of a wearer by a headgear having one or more straps adapted to fit over/around the patient's head. Because such respiratory patient interface devices are typically worn for an extended period of time, it is important for the headgear to maintain the patient interface in a desired position while doing so in a manner that is comfortable to the patient.

It is also desirable, however, that the respiratory patient interface device be relatively easy for the patient to install on the head. Previous devices that have been comfortable for the patient for extended periods and that maintain a reliable seal on the patient's face for extended periods have typically been relatively complicated devices that have been somewhat difficult to install. It thus would be desirable to provide an improved patient interface.

### SUMMARY OF THE INVENTION

In certain embodiments, the general nature of the invention can be stated as including resilient cushion that is structured to engage the face of a patient and to form a seal between the face of the patient and a supply apparatus that is structured to provide a flow of breathing gas to an airway of the patient. The cushion can be generally stated as including a deformable bellows portion comprising a first deformable element and a second deformable element connected together at a vertex. The bellows portion extends about the perimeter of a region which, when the cushion is engaged with the face, is structured to be an enclosed region situated between the face and the supply apparatus. The first deformable element can be generally stated as including a first leg that extends from the vertex, with the first deformable element being structured to be engaged with the face of the patient. The second deformable element can be generally stated as including a second leg that extends from the vertex, with the second deformable element being structured to be engaged with the supply apparatus. At least one of the first leg and the second leg has formed therein an indentation that extends along at least a portion of the perimeter and that provides a localized region of reduced stiffness.

These and other objects, features, and characteristics of the present invention, as well as the methods of operation and functions of the related elements of structure and the combination of parts and economies of manufacture, will become more apparent upon consideration of the following description and the appended claims with reference to the accompanying drawings, all of which form a part of this specification, wherein like reference numerals designate corresponding parts in the various figures. It is to be expressly understood, however, that the drawings are for the purpose of illustration and description only and are not intended as a definition of the limits of the invention.

### BRIEF DESCRIPTION OF THE DRAWINGS

FIG. 1 is a front elevational view of an improved patient interface in accordance with the present invention;

FIG. 2 is a side elevational view of the patient interface of FIG. 1 installed on a patient;

FIG. 3 is an enlarged view of a portion of the patient interface of FIGS. 1 and 2, partially disassembled;

FIG. 4 is a perspective view of a clip of the patient interface of FIGS. 1 and 2;

FIG. 5 is a sectional view as taken along line 5-5 of FIG. 1;

FIG. 6 is a view similar to FIG. 1, except depicting a strap of a strap apparatus connected with a pair of posts of the patient interface;

FIG. 7A is a perspective view of an improved deformable cushion in accordance with the present invention of the patient interface of FIGS. 1 and 2;

FIG. 7B is another perspective view of the cushion of FIGS. 1 and 2;

FIG. 8 is a sectional view as taken along line 8-8 of FIG. 7B; and

FIG. 9 is an enlarged view of an indicated portion of FIG. 8.

### DETAILED DESCRIPTION OF EXEMPLARY EMBODIMENT

As used herein, the singular form of “a”, “an”, and “the” include plural references unless the context clearly dictates otherwise. As used herein, the statement that two or more parts or components are “coupled” shall mean that the parts are joined or operate together either directly or indirectly, i.e., through one or more intermediate parts or components, so long as a link occurs. As used herein, “directly coupled” means that two elements are directly in contact with each other. As used herein, “fixedly coupled” or “fixed” means that two components are coupled so as to move as one while maintaining a constant orientation relative to each other.

As used herein, the word “unitary” means a component is created as a single piece or unit. That is, a component that includes pieces that are created separately and then coupled together as a unit is not a “unitary” component or body. As employed herein, the statement that two or more parts or components “engage” one another shall mean that the parts exert a force against one another either directly or through one or more intermediate parts or components.

Directional phrases used herein, such as, for example and without limitation, top, bottom, left, right, upper, lower, front, back, and derivatives thereof, relate to the orientation of the elements shown in the drawings and are not limiting upon the claims unless expressly recited therein.

An improved patient interface 2 in accordance with an exemplary embodiment of the invention is depicted generally in FIGS. 1 and 2. In FIG. 2, patient interface 2 is depicted as being installed on the face of a patient 4. Patient interface 2 is advantageously configured to provide a flow of breathing gas to the airways of patient 4.

Patient interface 2 can be said to include a supply apparatus 6 that is connected with a source of breathable gas, a cushion 8, and a headgear 10. Cushion 8 is mounted to supply apparatus 6 and is configured to deformably engage a face of patient 4 and to form a seal between the face of patient 4 and supply apparatus 6. Headgear 10 is connectable with supply apparatus 6 and can be used to mount patient interface 2 on the head of patient 4.

As can be understood from FIGS. 1 and 2, supply apparatus 6 can be said to include a frame 12 that includes a lower portion 14 and an upper portion 16. Frame 12 is a relatively rigid member which may be formed out of an appropriate material, such as a polycarbonate or other relatively rigid plastic material or other materials. Supply apparatus 6 can further be said to include a connector 18 mounted on frame 12. As can be understood from FIG. 1, connector 18 is connected with a source of breathing gas 20 that supplies a flow of breathing gas to patient interface 2. Connector 18 is in fluid communication with source of breathing gas 20 via a hose that is not expressly depicted herein for purposes of simplicity of disclosure. The breathing gas that is provided to patient interface 2 can be any of a wide variety of gases including combinations of gases such as air or other combinations of gases.

Upper portion 16 of frame 12 includes a forehead brace 22 that is structured to engage the forehead of patient 4. The engagement of forehead brace 22 with the forehead of patient 4 provides additional support to frame 12 and thereby assists cushion 8 in maintaining a seal with the face of patient 4 in order to reliably provide the flow of breathing gas to the airways of patient 4. It is noted, however, that other embodiments of the patient interface can be configured to not include forehead brace 22 while still remaining within the scope of the present concept.

Headgear 10 is depicted in FIGS. 1 and 2 as including an upper strap 23 that is connected with forehead brace 22 and which can be connected with the upper regions of the head of patient 4. FIGS. 1 and 2 further depict headgear 10 as including a strap apparatus 24 which, in FIGS. 1 and 2, is depicted as being connected with lower portion 14 of frame 12 and which is depicted in FIG. 2 as extending about the head of patient 4. As will be set forth in greater detail below, strap apparatus 24 is connectable with supply apparatus 6 and is detachable therefrom to facilitate the installation of patient interface 2 on patient 4 and the removal therefrom in a fashion that is simple and convenient for patient 4.

More particularly, and as can be understood from FIG. 3, supply apparatus 6 further comprises a pair of supports 26A and 26B disposed at opposite sides of lower portion 14 of frame 12. Supports 26A and 26B each include a post 28A and 28B that is mounted to frame 12 with an upper brace 30A and 30B and a lower brace 31A and 31B. Upper and lower braces 30A, 30B, 31A, and 31B support posts 28A and 28B at positions spaced from frame 12 in such a fashion to provide a passageway 32A and 32B adjacent each post 28A and 28B. More particularly, passageway 32A can be said to extend generally between post 28A and frame 12 and between upper brace 30A and lower brace 31A. Similarly, passageway 32B extends between post 28B and frame 12 and additionally extends between upper and lower braces 30B and 31B.

Posts 28A and 28B are configured such that an external surface 33A and 33B is arcuate along at least a portion thereof. In the exemplary embodiment depicted herein, the arcuate portions of surfaces 33A and 33B are of a generally cylindrical shape which permits strap apparatus 24 to be movably connected with posts 28A and 28B, as will be explained in greater detail below. It is noted, however, that in other embodiments not expressly depicted herein, posts 28A and 28B potentially may have external surfaces that are of another type of arcuate configuration, such as a spherical shape and the like depending upon the needs of the particular application.

As can be understood from FIGS. 1 and 2, strap apparatus 24 can be said to include a strap 34 and a pair of clips 36A and 36B. Clips 36A and 36B are connectable with strap 34 at the opposite ends thereof, as is depicted generally in FIG. 1. For example strap 36 could include hook and loop fasteners or other appropriate structures at its ends to enable clips 36A and 36B to be mounted thereon. An underside of clip 36A is depicted in greater detail in FIG. 4.

More particularly, FIG. 4 depicts clip 36A as including a housing 38 having a receptacle 40 formed therein that is structured to removably receive post 28A. Receptacle 40 can be seen as including an arcuate reception surface 42 situated opposite a lug 44 and between which post 28A can be received, as is depicted generally in FIG. 5. Since reception surface 42 is shaped to have an arcuate configuration that corresponds with that of the cylindrical shape of surface 33A of post 28A, clip 36A is at least somewhat pivotable with respect to post 28A when post 28A is received in receptacle 40. Clip 36A further includes a strut 46 that extends between opposed portions of housing 38 and which is structured to be connected with an end of strap 34, as is indicated in FIG. 5.

User 4 can apply a compressive force to clips 36A and 36B to press them onto posts 28A and 28B, respectively, with strap 34 extending around the rear of the head of patient 4. This causes posts 28A and 28B to be received in receptacles 40 of clips 36A and 36B. In so doing, tactile and audible feedback are provided by posts 28A and 28B being received against reception surfaces 42. Such feedback facilitates installation since patient 4 can be assured that patient interface 2 is properly assembled. Clips 36A and 36B can be easily removed from posts 28A and 28B by patient 4 applying forces to pull clips 36A and 36B away from frame 12, which dislodges posts 28A and 28B from receptacles 40 and enables removal of patient interface 2 from patient 4.

Since clips 36A and 36B are mounted to the ends of strap 34 with, for instance, hook and loop fasteners or other structures on strap 34, patient interface 2 can be mounted on patient 4 by receiving clips 36A and 36B on posts 28A and 28B, respectively, without a need of further adjustment of headgear 10. That is, once patient interface 2 is properly mounted on patient 4 for the first time, with strap 34 being adjusted on clips 36A and 36B to a state of appropriate fit, clips 36A and 36B can be removed from and reattached to supply apparatus 6 without a need of readjusting headgear 10 after each installation. This promotes simple and efficient installation and removal of patient interface 2, which is advantageous. The audible and tactile feedback of clips 36A and 36B when posts 28A and 28B are received in receptacles 40 further promotes ease of installation since patient 4 can be assured that patient interface 2 is properly installed.

Further advantageously, and as is indicated generally in FIG. 6, strap 34 can alternatively be mounted directly to posts 28A or 28B or both in the event that clips 36A or clip 36B or both should become broken or lost. That is, FIG. 6 depicts posts 28A and 28B having the opposite ends of strap 34 mounted thereon by passing the ends of strap 34 through passageways 32A and 32B and by fastening the ends of strap 34 back on themselves with the aforementioned hook and loop fasteners or other structures. Such connection between an end of strap 34 and post 28A and another connection of an opposite end of strap 34 with post 28B enables the connections of strap 34 to be movable with respect to posts 28A and 28B, much in the fashion whereby clip 36A was movably mounted to post 28A.

It thus can be seen that posts 28A and 28B are configured to enable the ends of strap 34 to be mounted directly thereon in the event that clips 36A or 36B or both should become unavailable. This is advantageous since a potential typically exists that either or both of clips 36A and 36B may be lost, broken, etc. If such unavailability of clips 36A or 36B or both should occur at bedtime, patient 4 can mount an end of strap 34 directly to posts 28A or 28B or both in order that patient interface 2 can be mounted on patient 4 for the night to provide a flow of breathing gas to patient 4. Patient 4 can thereafter order replacement components, as needed, or potentially can continue to mount strap 34 directly to posts 28A or 28B or both for an indefinite period of time depending upon the needs of the particular situation. By providing supports 26A and 26B with the versatility to enable connection of strap apparatus 24 alternative thereto with clips 36A and 36B or with strap 34 itself directly on posts 28A or 28B or both, patient interface 2 can be made more reliable for patient 4. That is, patient interface 2 can be made to be usable by patient 4 even in the event that certain components thereof may become broken, lost, or otherwise unavailable. Such versatility improves the usability of patient interface 2 and increases the likelihood of therapeutic treatment that is provided to patient 4.

Cushion 8 is further depicted in FIGS. 7A-9. In accordance with the present invention, cushion 8 is advantageously configured to provide an enhanced fit on patient 4 to more reliably form a seal between the face of patient 4 and supply apparatus 6. Cushion 8 is formed of a resilient, deformable material such as a silicone, rubber, or other material, by way of example. The seal formed between cushion 8 and the face of patient 4 resists leakage of the flow of breathing gas between cushion 8 and the face of patient 4, which promotes the flow of breathing gas being supplied to the airways of patient 4 rather than leaking from patient interface 2.

Cushion 8 can be said to include a bellows portion 48 that is deformable and that is collapsible in the fashion of a convolution of a convoluted deformable structure. Bellows portion 48 can be said to include a first deformable portion 50 that is engageable with the face of patient 4 to form a seal therewith. Bellows portion 48 can be said to further include a second deformable portion 52 that is connectable with supply apparatus 6. In the exemplary embodiment depicted herein, cushion 8 is molded to frame 12 to connect a peripheral region of frame 12 with second deformable portion 52. More particularly, and as can be understood from FIGS. 7A-8, second deformable portion 52 can be said to further include an attachment element 64 that is connected with frame 12 via, for example, the aforementioned molding operation, although such attachment is not expressly depicted in FIGS. 7A-9 for purposes of simplicity of disclosure. Other formation methodologies can be employed without departing from the present concept.

First and second deformable portions 50 and 52 are joined with one another at a vertex 54 that is indicated generally in FIGS. 8 and 9. More particularly, first deformable portion 50 includes a first leg 56 and a patient engagement element 58 that are connected together. As can be seen in FIG. 9, first leg 56 can be said to extend in a direction generally away from vertex 54 and to have a length 60 measured in a direction generally away from vertex 54. Second deformable portion 52 includes a second leg 62 that can likewise be said to extend away from vertex 54 and to have a length 66 measured in a direction generally away from vertex 54. Length 60 of first leg 56 is greater than length 66 of second leg 62, whereby second leg 62 can be said to have a greater stiffness, i.e., spring constant, than that of first leg 56.

As can be understood from FIG. 7A, cushion 8 can be said to enclose a region 68 which, when patient interface 2 is installed on patient 4, is enclosed within cushion 8 and is disposed between supply apparatus 6 and the face of patient 4. More particularly, cushion 8 can be said to extend about a perimeter of region 68.

In accordance with the present invention, second leg 62 has formed therein an indentation 70 that provides a region of reduced stiffness which can be said to be localized in the region of the indentation 70. That is, it can be seen from FIG. 9 that first leg 56 and second leg 62 each have nominal thicknesses (i.e., in a direction transverse to lengths 60 and 66 and into to plane of the page of FIG. 9) that are roughly similar. As such, the relatively shorter length 66 of second leg 62 compared with the relatively longer 60 of first leg 56 indicates that second leg 62 would generally be stiffer in bending with respect to vertex 54 than first leg 56. However, since second leg 62 has indentation 70 formed therein, indentation 70 reduces the thickness of second leg 62 at a location generally between vertex 54 and attachment element 64 to provide a region of reduced stiffness at the location of indentation 70. Second leg 62 thus has enhanced localized deformability in the vicinity of indentation 70, and such enhanced deformability improves the fit of cushion 8 on the face of patient 4.

Cushion 8 can be said to include an apex region 74 that is shown in FIGS. 7A and 7B. Apex region 72 can be said to extend generally across the bridge of the nose patient 4 when patient interface is installed on patient 4 in a fashion demonstrated generally in FIG. 2. As can be understood from FIG. 7B, indentation 70 extends between a pair of terminations of 72A and 72B disposed on opposite sides of cushion 8. Since apex region 72 is situated generally across the bridge of the nose of patient 4 when patient interface 2 is installed on patient 4, it can be understood that the portions of indentation 70 that extend between apex region 74 and terminations 72A and 72B are situated adjacent the sides of the nose of patient 4 when patient interface 2 is installed on patient 4.

As can be best understood from FIG. 9, indentation 70 is of a width 76 that is measured in a direction generally away from vertex 54, i.e., parallel to the direction of length 66 of second leg 62. Indentation 70 can also be said to have a depth 78 in a direction transverse to width 76. FIG. 7B depicts indentation 70 as having its width 76 and depth 78 at their maximum dimensions in the vicinity of apex region 74. Moreover, and as can be understood from FIGS. 7A-8, at least one of width 76 and depth 78 gradually decreases as indentation 70 extends in a direction from apex region 74 generally toward terminations 72A and 72B. In the exemplary embodiment depicted herein, both width 76 and depth 78 decrease along the length of indentation 70 in a direction generally away from apex region 74 toward the terminations 72A and 72B.

It thus can be understood that by providing indentation 70 in the vicinity of the bridge of the nose of patient 4 and the sides of the nose of patient 4, an enhanced fit between cushion 8 and the nose of patient 4 is provided. Such enhanced fit is provided by the localized region of reduced stiffness, i.e., region of increased compliance, in the vicinity of indentation 70. That is, when cushion 8 is received on the face of patient 4, first and second deformable portions 50 and 52 themselves may deform, plus second leg 62 further deforms at the region of enhanced compliance afforded by indentation 70. This provides an ability to accommodate a greater variety of nose geometries than was previously possible. Such improved fit increases the comfort to patient 4 and improves the reliability of the seal that is formed between cushion 8 and patient 4, which is desirable.

It is noted that in other embodiments of cushion 8, indentation 70 can be of other configurations, such as having different dimensions or being positioned in a different location. Moreover, it is understood that more than one indentation can be formed on cushion 8, and such indentations can be formed on either or both of first and second legs 56 and 62 depending upon the needs of the particular application. It thus can be seen that any desired type of localized region of reduced stiffness can be provided on cushion 8 depending upon the needs of the particular application. It thus can be understood that indentation 70 is merely one example of a single instance of a localized region of reduced stiffness, i.e., increased compliance, and that cushion 8 can be configured in other embodiments to include any number of such regions of reduced stiffness in any of a variety of locations thereon depending upon the needs of the particular application.

In the claims, any reference signs placed between parentheses shall not be construed as limiting the claim. The word “comprising” or “including” does not exclude the presence of elements or steps other than those listed in a claim. In a device claim enumerating several means, several of these means may be embodied by one and the same item of hardware. The word “a” or “an” preceding an element does not exclude the presence of a plurality of such elements. In any device claim enumerating several means, several of these means may be embodied by one and the same item of hardware. The mere fact that certain elements are recited in mutually different dependent claims does not indicate that these elements cannot be used in combination.

Although the invention has been described in detail for the purpose of illustration based on what is currently considered to be the most practical and preferred embodiments, it is to be understood that such detail is solely for that purpose and that the invention is not limited to the disclosed embodiments, but, on the contrary, is intended to cover modifications and equivalent arrangements that are within the spirit and scope of the appended claims. For example, it is to be understood that the present invention contemplates that, to the extent possible, one or more features of any embodiment can be combined with one or more features of any other embodiment.

1. A resilient cushion that is structured to engage the face of a patient and to form a seal between the face of the patient and a supply apparatus that is structured to provide a flow of breathing gas to an airway of the patient, the cushion comprising:
a deformable bellows portion comprising a first deformable element and a second deformable element connected together at a vertex, the bellows portion extending about the perimeter of a region which, when the cushion is engaged with the face, is structured to be an enclosed region situated between the face and the supply apparatus;
the first deformable element comprising a first leg that extends from the vertex, the first deformable element being structured to be engaged with the face of the patient;
the second deformable element comprising a second leg that extends from the vertex, the second deformable element being structured to be engaged with the supply apparatus; and
at least one of the first leg and the second leg having formed therein an indentation that extends along at least a portion of the perimeter and that provides a localized region of reduced stiffness that is localized along the perimeter at a location that is structured to be situated generally in the vicinity of the bridge of the nose.

2. The cushion of claim 1, wherein the indentation extends along at least a portion of the perimeter that is structured to be situated adjacent the nose of the patient.

3. The cushion of claim 2, wherein the indentation extends along a portion of the perimeter that is structured to extend across the bridge of the nose and adjacent the sides of nose.

4. The cushion of claim 1, wherein the first leg has a length measured in a direction extending generally away from the vertex and wherein the second leg has another length measured in a direction extending generally away from the vertex, the length being greater than the another length, the indentation being formed in the second leg.

5. The cushion of claim 4, wherein the second deformable element comprises an attachment element disposed on the second leg opposite the vertex, the attachment element being structured to be connected with the supply apparatus, the indentation being situated between the vertex and the attachment element.

6. The cushion of claim 1, wherein the bellows portion comprises an apex region that is structured to extend across the bridge of the nose of the patient, and wherein the indentation has a width measured in a direction extending generally away from the vertex and a depth measured in a direction extending generally transverse to the width, at least one of the width and the depth decreasing in a direction extending along the perimeter generally away from the apex region.

7. The cushion of claim 6, wherein the indentation extends between a pair of terminations situated at alternate sides of the apex region, and wherein the width and the depth both decrease in a direction extending along the perimeter generally away from the apex region and toward of the terminations.

8. A patient interface that comprises the cushion of claim 1 and that is structured to be engaged with the face of a patient and to provide a flow of breathing gas to an airway of the patient, the patient interface further comprising:
a supply apparatus that is structured to be connected with a source of breathing gas and that comprises at least a first support which comprises a post; and
a strap apparatus that is structured to extend around at least a portion of the patient's head, the strap apparatus comprising a strap and at least a first clip, the at least first clip being mountable on an end of the strap, the at least first clip and the end of the strap being alternatively connectable with the post to enable the patient interface to be mounted on the patient.
